(19) United States
(12) Patent Application Publication
(10) Pub. No.: US 2022/0125381 A1
(43) Pub. Date: Apr. 28, 2022

(54) MEDICAL DEVICES AND METHODS FOR MONITORING AND TREATMENT WITH SYNTHETIC POLYMERS EXHIBITING SPECIFIC BINDING

(71) Applicant: Boston Scientific Scimed, Inc., Maple Grove, MN (US)

(72) Inventor: Paul Vincent Grosso, Maple Grove, MN (US)

(21) Appl. No.: 17/507,982

(22) Filed: Oct. 22, 2021

Related U.S. Application Data
(60) Provisional application No. 63/104,781, filed on Oct. 23, 2020.

(51) Int. Cl. A61B 5/00; A61M 1/36; A61M 1/38; B01D 39/16

(52) U.S. Cl. CPC A61B 5/6866; A61M 1/3607; B01D 2323/39; A61M 1/38; B01D 39/1623; A61M 1/3621

(57) ABSTRACT

Embodiments herein relate to medical devices and methods for monitoring and/or treatment including the use of synthetic polymers exhibiting specific binding for compounds such as disease state markers or toxic substances. In an embodiment, a method of testing a patient for a disease state is included, the method can include withdrawing a fluid sample from the patient and contacting the fluid sample with an extracorporeal monitoring device. The extracorporeal monitoring device can include a microporous membrane. The microporous membrane can include a synthetic polymer, wherein the synthetic polymer exhibits binding specificity with a disease state marker. The method can further include evaluating the extracorporeal monitoring device for the presence of the disease state marker. Other embodiments are included herein.

This application claims the benefit of U.S. Provisional Application No. 63/104,781, filed Oct. 23, 2020, the content of which is herein incorporated by reference in its entirety.

### FIELD

Embodiments herein relate to medical devices and methods. More specifically, embodiments herein relate to medical devices and methods for monitoring and/or treatment including the use of synthetic polymers exhibiting specific binding for compounds such as disease state markers or toxic substances.

### BACKGROUND

According to the American Cancer Society, cancer accounts for nearly 25% of the deaths that occur in the United States each year. Cancer can form if one normal cell in any part of the body mutates and then begins to grow and multiply too much and too quickly. Cancer can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, cancerous tumors are the result of unchecked rapid cellular division.

The current standard of care for cancer can include first-line therapies such as surgery, radiation therapy, and chemotherapy. Additional second-line therapies can include radioactive seeding, cryotherapy, hormone or biologics therapy, ablation, and the like. Combinations of first-line therapies and second-line therapies can also be a benefit to patients if one particular therapy on its own is not effective.

However, current cancer therapies are less than perfect. For example, many therapies have substantial side-effects. For example, various chemotherapeutic agents can be toxic substances. Because of side effects, in some cases, an approach of “watchful waiting” can be pursued. This is particularly true with slow-developing cancers like prostate cancer. However, “watchful waiting” increases the need for monitoring the progress of the cancer.

Further, with some therapies, such as with surgical interventions or ablation, it can be difficult to ensure that all the cancerous tissue has been removed or destroyed.

### SUMMARY

Embodiments herein relate to medical devices and methods for monitoring and/or treatment including the use of synthetic polymers exhibiting specific binding for compounds such as disease state markers or toxic substances.

In a first aspect, a method of testing a patient for a disease state is included, the method including withdrawing a fluid sample from the patient and contacting the fluid sample with an extracorporeal monitoring device. The extracorporeal monitoring device can include a fibrous substrate. The fibrous substrate can include a synthetic polymer, wherein the synthetic polymer exhibits binding specificity with a disease state marker. The method can further include evaluating the extracorporeal monitoring device for the presence of the disease state marker.

In a second aspect, in addition to one or more of the preceding or following aspects, or in the alternative to some aspects, the disease state marker includes at least one of an oncological marker, a cardiovascular disease marker, and a neurological disease marker.

In a third aspect, in addition to one or more of the preceding or following aspects, or in the alternative to some aspects, the fibrous substrate can include electrospun fibers.

In a fourth aspect, in addition to one or more of the preceding or following aspects, or in the alternative to some aspects, the fibrous substrate can include fibers of electrospun hydrogel.

In a fifth aspect, in addition to one or more of the preceding or following aspects, or in the alternative to some aspects, the fibrous substrate can include fibers of electrospun crosslinked hydrogel.

In a sixth aspect, in addition to one or more of the preceding or following aspects, or in the alternative to some aspects, the synthetic polymer can include a hydrogel.

In a seventh aspect, in addition to one or more of the preceding or following aspects, or in the alternative to some aspects, the synthetic polymer can include a copolymer including n-isopropyl acrylamide and acrylic acid.

In an eighth aspect, in addition to one or more of the preceding or following aspects, or in the alternative to some aspects, the extracorporeal monitoring device can include a flow channel.

In a ninth aspect, in addition to one or more of the preceding or following aspects, or in the alternative to some aspects, the fibrous substrate is disposed within or surrounds the flow channel.

In a tenth aspect, a method of testing a patient for a disease state is included, the method can include withdrawing a fluid sample from the patient and contacting the fluid sample with an extracorporeal monitoring device. The extracorporeal monitoring device can include a microporous membrane. The microporous membrane can include a synthetic polymer, wherein the synthetic polymer exhibits binding specificity with a disease state marker. The method can further include evaluating the extracorporeal monitoring device for the presence of the disease state marker.

In an eleventh aspect, in addition to one or more of the preceding or following aspects, or in the alternative to some aspects, the disease state marker includes at least one of an oncological marker, a cardiovascular disease marker, and a neurological disease marker.

In a twelfth aspect, in addition to one or more of the preceding or following aspects, or in the alternative to some aspects, the microporous membrane can include electrospun fibers.

In a thirteenth aspect, in addition to one or more of the preceding or following aspects, or in the alternative to some aspects, the synthetic polymer can include a hydrogel.

In a fourteenth aspect, in addition to one or more of the preceding or following aspects, or in the alternative to some aspects, the synthetic polymer can include a copolymer including n-isopropyl acrylamide and acrylic acid.

In a fifteenth aspect, in addition to one or more of the preceding or following aspects, or in the alternative to some aspects, the extracorporeal monitoring device can include a flow channel.

In a sixteenth aspect, in addition to one or more of the preceding or following aspects, or in the alternative to some aspects, the microporous membrane is disposed within or surrounds the flow channel.

In a seventeenth aspect, a method of testing a patient for cancer is included, the method including withdrawing a fluid sample from the patient and contacting the fluid sample with an extracorporeal monitoring device. The extracorporeal monitoring device can include a fibrous substrate. The fibrous substrate can include a synthetic polymer, wherein the synthetic polymer exhibits binding specificity with an oncological marker. The method can further include evaluating the extracorporeal monitoring device for the presence of at least one of the oncological marker and an exosome bearing the oncological marker.

In an eighteenth aspect, in addition to one or more of the preceding or following aspects, or in the alternative to some aspects, the oncological marker includes at least one of VEGF, CD63, annexin A6, and mesothelin.

In a nineteenth aspect, in addition to one or more of the preceding or following aspects, or in the alternative to some aspects, the fibrous substrate can include electrospun fibers.

In a twentieth aspect, in addition to one or more of the preceding or following aspects, or in the alternative to some aspects, the synthetic polymer can include a hydrogel.

In a twenty-first aspect, in addition to one or more of the preceding or following aspects, or in the alternative to some aspects, the synthetic polymer can include a copolymer including n-isopropyl acrylamide and acrylic acid.

In a twenty-second aspect, in addition to one or more of the preceding or following aspects, or in the alternative to some aspects, the extracorporeal monitoring device can include a flow channel.

In a twenty-third aspect, in addition to one or more of the preceding or following aspects, or in the alternative to some aspects, the fibrous substrate is disposed within or surrounds the flow channel.

In a twenty-fourth aspect, a method of removing a substance from fluids of a patient is included, the method including withdrawing a fluid sample from the patient, and contacting the fluid sample with an extracorporeal filtration device. The extracorporeal filtration device can include a fibrous substrate. The fibrous substrate can include a synthetic polymer, wherein the synthetic polymer exhibits binding specificity with the substance to be removed. The method can further include returning filtered fluids to the patient.

In a twenty-fifth aspect, in addition to one or more of the preceding or following aspects, or in the alternative to some aspects, the fibrous substrate can include electrospun fibers.

In a twenty-sixth aspect, in addition to one or more of the preceding or following aspects, or in the alternative to some aspects, the synthetic polymer can include a hydrogel.

In a twenty-seventh aspect, in addition to one or more of the preceding or following aspects, or in the alternative to some aspects, the synthetic polymer can include a copolymer including n-isopropyl acrylamide and acrylic acid.

In a twenty-eighth aspect, in addition to one or more of the preceding or following aspects, or in the alternative to some aspects, the extracorporeal filtration device can include a flow channel.

In a twenty-ninth aspect, in addition to one or more of the preceding or following aspects, or in the alternative to some aspects, the fibrous substrate is disposed within or surrounds the flow channel.

In a thirtieth aspect, in addition to one or more of the preceding or following aspects, or in the alternative to some aspects, the substance can include at least one of a toxic substance or a chemotherapy drug.

In a thirty-first aspect, in addition to one or more of the preceding or following aspects, or in the alternative to some aspects, the substance can include an oncological marker or an exosome bearing the same.

In a thirty-second aspect, in addition to one or more of the preceding or following aspects, or in the alternative to some aspects, the substance can include an agent responsible for the growth and/or spread of a cancer.

In a thirty-third aspect, a method of evaluating an oncological surgical site is included, the method including applying a visualization device to the oncological surgical site. The visualization device can include a fibrous substrate and a synthetic polymer. The synthetic polymer can exhibit specific binding to an oncological marker and can be integrated on or into the fibrous substrate. The visualization device can also include a visualization element, wherein the visualization element is bonded to at least one of the synthetic polymer and the fibrous substrate. The method can further include imaging the visualization device.

In a thirty-fourth aspect, in addition to one or more of the preceding or following aspects, or in the alternative to some aspects, the oncological marker includes at least one of VEGF, CD63, annexin A6, and mesothelin.

In a thirty-fifth aspect, in addition to one or more of the preceding or following aspects, or in the alternative to some aspects, the visualization element can include a fluorescent dye.

In a thirty-sixth aspect, in addition to one or more of the preceding or following aspects, or in the alternative to some aspects, the fibrous substrate can include electrospun fibers.

In a thirty-seventh aspect, in addition to one or more of the preceding or following aspects, or in the alternative to some aspects, the electrospun fibers are formed at least partly from the synthetic polymer.

In a thirty-eighth aspect, in addition to one or more of the preceding or following aspects, or in the alternative to some aspects, wherein imaging the visualization device further includes applying ultraviolet light.

In a thirty-ninth aspect, in addition to one or more of the preceding or following aspects, or in the alternative to some aspects, the visualization device is a flexible, planar article.

In a fortieth aspect, in addition to one or more of the preceding or following aspects, or in the alternative to some aspects, the visualization device is a flexible pouch.

In a forty-first aspect, in addition to one or more of the preceding or following aspects, or in the alternative to some aspects, the visualization device is a deformable amorphous mass.

In a forty-second aspect, in addition to one or more of the preceding or following aspects, or in the alternative to some aspects, the synthetic polymer can include a hydrogel.

In a forty-third aspect, in addition to one or more of the preceding or following aspects, or in the alternative to some aspects, the synthetic polymer can include a copolymer including n-isopropyl acrylamide and acrylic acid.

In a forty-fourth aspect, in addition to one or more of the preceding or following aspects, or in the alternative to some aspects, imaging the visualization device can include identifying and least one of exosomes and oncological markers bound to the visualization device.

In a forty-fifth aspect, a method of monitoring cancerous tumor progression is included, the method can include placing an implantable monitoring device within a patient. The implantable monitoring device can include a fibrous substrate, and a synthetic polymer, wherein the synthetic polymer exhibits specific binding to an oncological marker. The synthetic polymer can be integrated on or into the fibrous substrate. The implantable monitoring device can define a fluid flow channel and the synthetic polymer can be disposed within the flow channel. The method can further include removing the implantable monitoring device from the patient and analyzing the implantable monitoring device for the presence of the oncological marker.

In a forty-sixth aspect, in addition to one or more of the preceding or following aspects, or in the alternative to some aspects, the method can further include analyzing the implantable monitoring device for the presence of exosomes bearing the oncological marker.

In a forty-seventh aspect, in addition to one or more of the preceding or following aspects, or in the alternative to some aspects, the fibrous substrate can include electrospun fibers.

In a forty-eighth aspect, in addition to one or more of the preceding or following aspects, or in the alternative to some aspects, the electrospun fibers are formed at least partly from the synthetic polymer.

In a forty-ninth aspect, in addition to one or more of the preceding or following aspects, or in the alternative to some aspects, the oncological marker includes at least one of VEGF, CD63, annexin A6, and mesothelin.

In a fiftieth aspect, in addition to one or more of the preceding or following aspects, or in the alternative to some aspects, the synthetic polymer can include a hydrogel.

In a fifty-first aspect, in addition to one or more of the preceding or following aspects, or in the alternative to some aspects, the synthetic polymer can include a copolymer including n-isopropyl acrylamide and acrylic acid.

In a fifty-second aspect, in addition to one or more of the preceding or following aspects, or in the alternative to some aspects, the implantable monitoring device is a flexible, planar article.

In a fifty-third aspect, in addition to one or more of the preceding or following aspects, or in the alternative to some aspects, the implantable monitoring device is a flexible pouch.

In a fifty-fourth aspect, in addition to one or more of the preceding or following aspects, or in the alternative to some aspects, the implantable monitoring device is a deformable amorphous mass.

In a fifty-fifth aspect, in addition to one or more of the preceding or following aspects, or in the alternative to some aspects, the implantable monitoring device defines a lumen.

In a fifty-sixth aspect, in addition to one or more of the preceding or following aspects, or in the alternative to some aspects, the implantable monitoring device can include a housing.

In a fifty-seventh aspect, in addition to one or more of the preceding or following aspects, or in the alternative to some aspects, the housing can include a flexible polymeric housing.

In a fifty-eighth aspect, a method of evaluating a tissue of a patient is included, the method including placing a biopsy device within a patient. The biopsy device can include a shaft and a tissue sampling element. The tissue sampling element can include a fibrous substrate. The fibrous substrate can include a synthetic polymer, wherein the synthetic polymer exhibits specific binding to a disease state marker. The method can further include removing the biopsy device from the patient and analyzing the tissue sampling element for the presence of at least one of the disease state marker and an exosome bearing the disease state marker.

In a fifty-ninth aspect, in addition to one or more of the preceding or following aspects, or in the alternative to some aspects, the disease state marker includes an oncological marker can include at least one of VEGF, CD63, annexin A6, and mesothelin.

In a sixtieth aspect, in addition to one or more of the preceding or following aspects, or in the alternative to some aspects, the synthetic polymer can include a hydrogel.

In a sixty-first aspect, in addition to one or more of the preceding or following aspects, or in the alternative to some aspects, the synthetic polymer can include a copolymer including n-isopropyl acrylamide and acrylic acid.

In a sixty-second aspect, in addition to one or more of the preceding or following aspects, or in the alternative to some aspects, the fibrous substrate can include electrospun fibers.

In a sixty-third aspect, in addition to one or more of the preceding or following aspects, or in the alternative to some aspects, the electrospun fibers are formed at least partly from the synthetic polymer.

In a sixty-fourth aspect, a extracorporeal medical device is included having a housing and a sensing substrate. The sensing substrate can include a synthetic polymer, wherein the synthetic polymer exhibits specific binding with at least one of a disease state marker, an exosome bearing the disease state marker, and a toxic substance. The sensing substrate can be disposed within the housing and can be configured to contact a fluid sample of a patient.

In a sixty-fifth aspect, in addition to one or more of the preceding or following aspects, or in the alternative to some aspects, the disease state marker includes at least one of an oncological marker, a cardiovascular disease marker, and a neurological disease marker.

In a sixty-sixth aspect, in addition to one or more of the preceding or following aspects, or in the alternative to some aspects, the oncological marker includes at least one of VEGF, CD63, annexin A6, and mesothelin.

In a sixty-seventh aspect, in addition to one or more of the preceding or following aspects, or in the alternative to some aspects, the toxic substance includes a chemotherapeutic agent.

In a sixty-eighth aspect, in addition to one or more of the preceding or following aspects, or in the alternative to some aspects, the sensing substrate can include a fibrous substrate.

In a sixty-ninth aspect, in addition to one or more of the preceding or following aspects, or in the alternative to some aspects, the fibrous substrate can include electrospun fibers.

In a seventieth aspect, in addition to one or more of the preceding or following aspects, or in the alternative to some aspects, the electrospun fibers can include a crosslinked hydrogel.

In a seventy-first aspect, in addition to one or more of the preceding or following aspects, or in the alternative to some aspects, the sensing substrate can include a microporous membrane.

In a seventy-second aspect, in addition to one or more of the preceding or following aspects, or in the alternative to some aspects, the sensing substrate can include a microporous monolith.

In a seventy-third aspect, in addition to one or more of the preceding or following aspects, or in the alternative to some aspects, the synthetic polymer can include a copolymer including n-isopropyl acrylamide and acrylic acid.

In a seventy-fourth aspect, in addition to one or more of the preceding or following aspects, or in the alternative to some aspects, the housing can include a flow channel. The flow channel can include an inner surface.

In a seventy-fifth aspect, in addition to one or more of the preceding or following aspects, or in the alternative to some aspects, the synthetic polymer lines the inner surface.

In a seventy-sixth aspect, in addition to one or more of the preceding or following aspects, or in the alternative to some aspects, the synthetic polymer is packed in the flow channel.

In a seventy-seventh aspect, in addition to one or more of the preceding or following aspects, or in the alternative to some aspects, the device further can include a sampling tube, wherein the sampling tube is in fluid communication with the housing.

In a seventy-eighth aspect, in addition to one or more of the preceding or following aspects, or in the alternative to some aspects, the device further can include a fluid pump, wherein the fluid pump is configured to cause the fluid sample to flow through the housing.

In a seventy-ninth aspect, in addition to one or more of the preceding or following aspects, or in the alternative to some aspects, the device further can include a fluid return tube, wherein the fluid return tube is in fluid communication with the housing.

In an eightieth aspect, an implantable monitoring device is included having a sensing substrate. The sensing substrate can include a synthetic polymer, wherein the synthetic polymer exhibits specific binding with at least one of a disease state marker, an exosome bearing the disease state marker, and a toxic substance. The sensing substrate can be configured to contact a fluid sample of a patient.

In an eighty-first aspect, in addition to one or more of the preceding or following aspects, or in the alternative to some aspects, the disease state marker includes at least one of an oncological marker, a cardiovascular disease marker, and a neurological disease marker.

In an eighty-second aspect, in addition to one or more of the preceding or following aspects, or in the alternative to some aspects, the oncological marker includes at least one of VEGF, CD63, annexin A6, and mesothelin.

In an eighty-third aspect, in addition to one or more of the preceding or following aspects, or in the alternative to some aspects, the toxic substance includes a chemotherapeutic agent.

In an eighty-fourth aspect, in addition to one or more of the preceding or following aspects, or in the alternative to some aspects, the implantable monitoring device is configured to be placed within a vein, an artery, a lymph duct, or a surgical site.

In an eighty-fifth aspect, in addition to one or more of the preceding or following aspects, or in the alternative to some aspects, the sensing substrate can include a fibrous substrate.

In an eighty-sixth aspect, in addition to one or more of the preceding or following aspects, or in the alternative to some aspects, the fibrous substrate can include electrospun fibers.

In an eighty-seventh aspect, in addition to one or more of the preceding or following aspects, or in the alternative to some aspects, the electrospun fibers can include a crosslinked hydrogel.

In an eighty-eighth aspect, in addition to one or more of the preceding or following aspects, or in the alternative to some aspects, the sensing substrate can include a microporous membrane.

In an eighty-ninth aspect, in addition to one or more of the preceding or following aspects, or in the alternative to some aspects, the sensing substrate can include a microporous monolith.

In a ninetieth aspect, in addition to one or more of the preceding or following aspects, or in the alternative to some aspects, the synthetic polymer can include a copolymer including n-isopropyl acrylamide and acrylic acid.

In a ninety-first aspect, in addition to one or more of the preceding or following aspects, or in the alternative to some aspects, the implantable monitoring device is a deformable amorphous mass.

In a ninety-second aspect, in addition to one or more of the preceding or following aspects, or in the alternative to some aspects, the implantable monitoring device is a flexible, planar article.

In a ninety-third aspect, in addition to one or more of the preceding or following aspects, or in the alternative to some aspects, the device further can include a housing.

In a ninety-fourth aspect, in addition to one or more of the preceding or following aspects, or in the alternative to some aspects, the housing can include a flexible polymeric material.

In a ninety-fifth aspect, in addition to one or more of the preceding or following aspects, or in the alternative to some aspects, the housing can include a flexible pouch.

In a ninety-sixth aspect, in addition to one or more of the preceding or following aspects, or in the alternative to some aspects, the sensing substrate is disposed within the housing.

In a ninety-seventh aspect, in addition to one or more of the preceding or following aspects, or in the alternative to some aspects, the housing can include a porous membrane.

In a ninety-eighth aspect, in addition to one or more of the preceding or following aspects, or in the alternative to some aspects, the housing can include a flow channel.

In a ninety-ninth aspect, in addition to one or more of the preceding or following aspects, or in the alternative to some aspects, the flow channel can include an inner surface.

In a one hundred and aspect, in addition to one or more of the preceding or following aspects, or in the alternative to some aspects, the synthetic polymer lines the inner surface of the flow channel.

In a one hundred and first aspect, in addition to one or more of the preceding or following aspects, or in the alternative to some aspects, the synthetic polymer is packed in the flow channel.

In a one hundred and second aspect, a biopsy device is included having a shaft and a tissue sampling element. The tissue sampling element can include a synthetic polymer. The tissue sampling element can be disposed on the shaft and wherein the synthetic polymer exhibits specific binding with at least one of a disease state marker, an exosome bearing the disease state marker, and a toxic substance.

In a one hundred and third aspect, in addition to one or more of the preceding or following aspects, or in the alternative to some aspects, the disease state marker includes at least one of an oncological marker, a cardiovascular disease marker, and a neurological disease marker.

In a one hundred and fourth aspect, in addition to one or more of the preceding or following aspects, or in the alternative to some aspects, the oncological marker includes at least one of VEGF, CD63, annexin A6, and mesothelin.

In a one hundred and fifth aspect, in addition to one or more of the preceding or following aspects, or in the alternative to some aspects, the toxic substance includes a chemotherapeutic agent.

In a one hundred and sixth aspect, in addition to one or more of the preceding or following aspects, or in the alternative to some aspects, the tissue sampling element can include a fibrous substrate.

In a one hundred and seventh aspect, in addition to one or more of the preceding or following aspects, or in the alternative to some aspects, the fibrous substrate can include electrospun fibers.

In a one hundred and eighth aspect, in addition to one or more of the preceding or following aspects, or in the alternative to some aspects, the electrospun fibers can include a crosslinked hydrogel.

In a one hundred and ninth aspect, in addition to one or more of the preceding or following aspects, or in the alternative to some aspects, the tissue sampling element can include a microporous membrane.

In a one hundred and tenth aspect, in addition to one or more of the preceding or following aspects, or in the alternative to some aspects, the tissue sampling element can include a microporous monolith.

In a one hundred and eleventh aspect, in addition to one or more of the preceding or following aspects, or in the alternative to some aspects, the synthetic polymer can include a copolymer including n-isopropyl acrylamide and acrylic acid.

In a one hundred and twelfth aspect, in addition to one or more of the preceding or following aspects, or in the alternative to some aspects, the shaft can include a flexible polymeric shaft.

In a one hundred and thirteenth aspect, in addition to one or more of the preceding or following aspects, or in the alternative to some aspects, the shaft can be configured to fit within an introducer sheath.

In a one hundred and fourteenth aspect, in addition to one or more of the preceding or following aspects, or in the alternative to some aspects, a method of testing a patient for a disease state is included. The method can include withdrawing a fluid sample from the patient, contacting the fluid sample with an extracorporeal monitoring device, the extracorporeal monitoring device comprising a fibrous substrate, the fibrous substrate comprising electrospun polymeric fibers with nanoparticles ionically or covalently bonded thereto, the nanoparticles comprising a synthetic polymer exhibiting binding specificity with a disease state marker, and evaluating the extracorporeal monitoring device for the presence of the disease state marker.

In a one hundred and fifteenth aspect, in addition to one or more of the preceding or following aspects, or in the alternative to some aspects, a method of testing a patient for cancer is included. The method can include a method of testing a patient for cancer including withdrawing a fluid sample from the patient, and contacting the fluid sample with an extracorporeal monitoring device. The extracorporeal monitoring device can include a fibrous substrate, the fibrous substrate comprising electrospun polymeric fibers with nanoparticles ionically or covalently bonded thereto, the nanoparticles comprising a synthetic polymer exhibiting binding specificity with an oncological marker. The method can also include evaluating the extracorporeal monitoring device for the presence of at least one of the oncological marker and an exosome bearing the oncological marker.

In a one hundred and sixteenth aspect, in addition to one or more of the preceding or following aspects, or in the alternative to some aspects, a method of testing a patient for cancer is included. The method can include removing a substance from fluids of a patient including withdrawing a fluid sample from the patient and contacting the fluid sample with an extracorporeal filtration device. The extracorporeal filtration device can include a fibrous substrate, the fibrous substrate comprising electrospun polymeric fibers with nanoparticles ionically or covalently bonded thereto, the nanoparticles comprising a synthetic polymer exhibiting binding specificity with the substance to be removed. The method can further include returning filtered fluids to the patient.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an exosome in accordance with various embodiments herein.

FIG. 2 is a schematic view of a fibrous substrate in accordance with various embodiments herein.

FIG. 3 is a schematic view of an approach for visualizing bound exosomes in accordance with various embodiments herein.

FIG. 4 is a schematic view of an electrospinning system in accordance with various embodiments herein.

FIG. 5 is a schematic view of a monitoring device in accordance with various embodiments herein.

FIG. 6 is a schematic view of a monitoring device in accordance with various embodiments herein.

FIG. 7 is a schematic view of a biopsy device in accordance with various embodiments herein.

FIG. 8 is a schematic view of an extracorporeal medical device in accordance with various embodiments herein.

FIG. 9 is a schematic view of an extracorporeal medical device in accordance with various embodiments herein.

FIG. 10 is a schematic view of an implantable monitoring device in accordance with various embodiments herein.

FIG. 11 is a cross-sectional view of an implantable monitoring device as taken along line 11-11′ of FIG. 10 in accordance with various embodiments herein.

FIG. 12 is a schematic view of a monitoring device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, an approach of “watchful waiting” can be pursued in the case of some disease states including certain forms of cancer. However, “watchful waiting” increases the need for monitoring the progress of the cancer. Further, with some therapies, such as with surgical interventions or ablation of diseased tissue, it can be difficult to ensure that all diseased tissue has been removed or destroyed. Thus, monitoring and detection systems relevant for specific disease states are highly useful. Further, it can be advantageous to selectively remove specific components (such as cancerous cells, cancerous cellular components, or the like) or substances (such as toxic substances or chemotherapeutic agents) from fluids of the body and then return the same in a cleansed state. Embodiments herein can address such needs and relate to medical devices (both implantable and extracorporeal) and methods for monitoring, detection, and/or treatment including the use of synthetic polymers exhibiting specific binding for compounds such as disease state markers or toxic substances.

The form of a material with desirable binding properties greatly impacts the ability to make use of the material in a device. For example, nanoparticles can be useful for some applications as they have an extremely high surface to volume ratio. However, nanoparticles may not be useful for integration into some types of devices, including implantable medical devices as retaining the nanoparticles can be difficult. Electrospun fibers generally have a very small diameter resulting in very high surface to volume ratios. For example, electrospun fibers can have diameters of less than 2000, 1000, 500, 250, 100, 50 or even 10 nanometers. However, not every polymer can be successfully electrospun and the process of electrospinning can impact the functional properties of a polymer. Significantly, however, it has been found herein that certain polymers with desirable binding properties can be electrospun and subsequently cross-linked while retaining the desirable binding properties. As such, embodiments herein include devices with polymeric electrospun fibrous mats that exhibit desirable binding properties and sufficient durability.

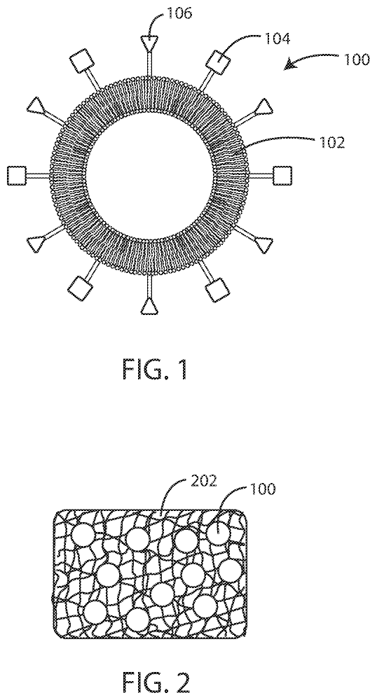

Referring now to FIG. 1, a schematic view of an exosome 100 is shown in accordance with various embodiments herein. The exosome 100 includes a phospholipid bilayer 102. The exosome 100 also includes a first antigen 104 (or first marker). The exosome 100 also includes a second antigen 106 (or second marker). While two antigens are shown on the surface of the exosome 100 in this illustration, it will be appreciated that exosomes can include any number of different antigens or markers. In some embodiments, the first antigen 104 or the second antigen 106 can be a disease state marker. In some embodiments, the first antigen 104 or the second antigen 106 can be an oncological marker, a cardiovascular disease marker, and a neurological disease marker. Further details regarding exemplary disease state markers are described in greater detail below.

In various embodiments herein, a fibrous substrate can be at least partially formed from a synthetic polymer that exhibits specific binding characteristics and the same can be included with a device. For example, a fibrous substrate can be at least partially formed from a synthetic polymer that exhibits specific binding characteristics for a disease state marker, a toxic substance, or a chemotherapeutic agent. Various devices can be formed taking advantage of specific binding characteristics including, but not limited to, implantable medical devices, implantable monitoring devices, extracorporeal devices, extracorporeal monitoring devices, extracorporeal clearance devices, biopsy devices, and the like.

The terms “specifically bind” and “specific binding,” as used herein, generally refer to the ability of a synthetic polymer herein to preferentially bind to a particular antigen or substance (such as a disease state marker, an oncological marker, a toxic substance, or a chemotherapeutic agent) that is present in a fluid or a tissue sample of patient. The specific binding interaction can discriminate between different antigens or substances in a sample, in some embodiments by more than about 10 to 100-fold or more (e.g., more than about 1,000 or 10,000-fold).

Referring now to FIG. 2, a schematic view of a fibrous substrate 202 is shown in accordance with various embodiments herein. In this view, a fibrous substrate 202 is shown along with exosomes 100 that are bound to the fibrous substrate 202. The exosomes including antigens/molecules thereon to which a synthetic polymer of the fibrous substrate 202 specifically binds. The fibrous substrate 202 can be a part of various medical devices including, but not limited to, implantable monitoring devices, extracorporeal devices, extracorporeal monitoring devices, extracorporeal clearance devices, biopsy devices, and the like.

In some cases, after markers, exosomes bearing markers, and/or toxic substances are bound to a fibrous substrate 202 or other structure herein that includes a synthetic polymer with specific binding characteristics, the same can be visualized using various techniques including, for example, microscopy, visualization dyes or compounds, and the like.

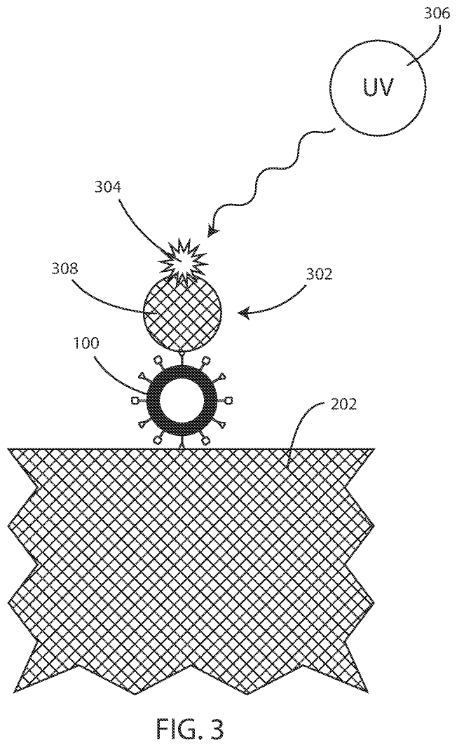

Referring now to FIG. 3, a schematic view of an approach for visualizing bound markers and/or exosomes including the same is shown in accordance with various embodiments herein. FIG. 3 shows an exosome 100 that is bound to a fibrous substrate 202. In this example, a visualization element 302 is also bound to the exosome 100. The visualization element 302 in this example can include a fluorescent dye 304 and a specific binding element 308. The specific binding element 308 can include a compound or structure that will specifically bind to a component on the exosome (which could be the same marker that the synthetic polymer of the fibrous substrate 202 is specific for or it could be a different component of the exosome or compounds/markers/antigens thereon). For example, after exosomes have bound to the fibrous substrate 202, a solution containing the visualization element 302 could be applied to the fibrous substrate 202 and then later the fibrous substrate could be rinsed to remove visualization elements 302 that are not specifically bound. In this example, the fluorescent dye 304 is one that fluoresces when being irradiated with UV light from a UV light source 306. For example, the fluorescent dye 304 could be a fluorescein dye. However, it will be appreciated there are many different ways for visualizing materials such as markers, exosomes bearing markers, and/or toxic substances that have bound to the fibrous substrate 202. In some cases, a visualization element 302 can be bonded to at least one of a synthetic polymer and a fibrous substrate 202.

A device including a fibrous substrate 202 such as that shown in FIGS. 2 and 3 can be used in various methods. For examples, in some embodiments herein, a method of testing a patient for a disease state can be included. The method can include withdrawing a fluid sample from the patient, and contacting the fluid sample with an extracorporeal monitoring device. The extracorporeal monitoring device can include a fibrous substrate 202 and the fibrous substrate 202 can include a synthetic polymer, wherein the synthetic polymer exhibits binding specificity with a disease state marker. The method can further include evaluating the extracorporeal monitoring device for the presence of the disease state marker. In various embodiments, evaluating the extracorporeal monitoring device can specifically include imaging the visualization device to identify exosomes bound to the visualization device. However, methods herein are not limited to the use of extracorporeal devices. For example, in some embodiments, a method can include inserting a monitoring device into or on a patient (e.g., an implantable device, a transitorily implantable device, a device draped on or over a patient such as a surgical site, etc.). The monitoring device can include a fibrous substrate 202 and the fibrous substrate 202 can include a synthetic polymer, wherein the synthetic polymer exhibits binding specificity with a disease state marker. The method can further include withdrawing the monitoring device from the patient and evaluating the monitoring device for the presence of the disease state marker.

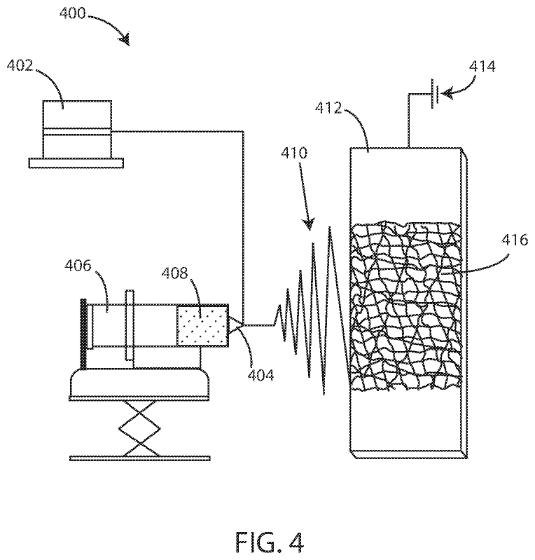

It will be appreciated that fibrous substrates herein can be formed in various ways. In some embodiments, fibrous substrates herein can be formed using an electrospinning process. Referring now to FIG. 4, a schematic view of an electrospinning system 400 is shown in accordance with various embodiments herein. The electrospinning system 400 can be used to create a fibrous substrate.

FIG. 4 shows a power supply 402 that provides the power to produce an electric field between a polymer composition 408 at a tip 404 of a syringe 406 and a deposition substrate 412. The deposition substrate 412 can be electrically grounded 414. The electric field created between the tip 404 and the deposition substrate 412 creates an electrostatic force that causes a surface tension of the droplet of the polymer composition 408 to be overcome. When the surface tension of the droplet of the polymer composition 408 is overcome by the electrostatic forces created, the droplet of the polymer composition 408 becomes a charged, continuous jet of electrospun fibers 410 that rapidly dry and thin in the air as the electrospun fibers 410 move toward the deposition substrate 412. The electrospun fibers 410 are deposited on the deposition substrate 412 as deposited fibers 416. In some embodiments, the deposited fibers 416 are arranged in a nonwoven, random orientation.

The deposited fibers 416 can have various diameters. In some embodiments, the diameter of the deposited fibers 416 can be less than 2000, 1000, 500, 250, 100, 50 or even 10 nanometers, or a diameter falling within a range between any of the foregoing. Thus, in various embodiments, the fibrous substrate 202 can include electrospun fibers. In various embodiments, the fibrous substrate 202 can specifically include fibers of electrospun hydrogel. In various embodiments, the fibrous substrate 202 can include fibers of electrospun crosslinked hydrogel. For example, after deposition, the material of the fibers can be crosslinked using various techniques including the use of chemical cross-linking agents (which may be present in the material being electrospun, but not activated to form cross links), irradiation based cross-linking, thermal cross-linking or the like.

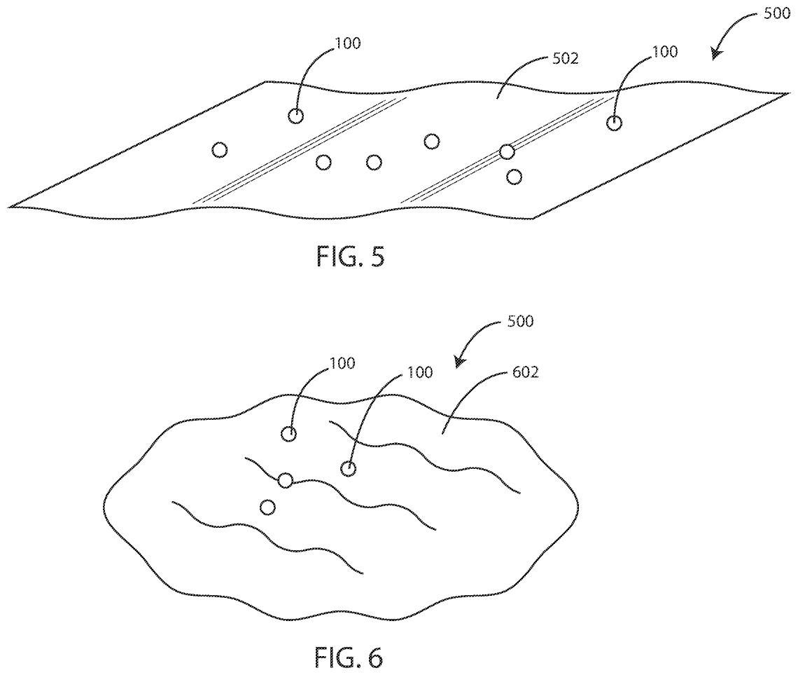

Monitoring devices herein (both extracorporeal devices as well as implantable, partially implantable, and transitorily implantable devices) can take on many different forms. Referring now to FIG. 5, a schematic view of a monitoring device 500 is shown in accordance with various embodiments herein. In this embodiment, the monitoring device 500 includes a flexible planar article or structure 502. FIG. 5 also shows an exosome 100 bound to the flexible planar structure 502. The monitoring device 500 in the form of the flexible planar structure 502 can be used in many different ways. In some embodiments, the monitoring device 500 can specifically be an implantable monitoring device. In other embodiments, the monitoring device 500 may be non-implanted.

In various embodiments, the implantable monitoring device 500 can be configured to be placed on or within a vein, an artery, a lymph duct, or a surgical site.

In various embodiments, the implantable monitoring device 500 can be a visualization device, such as a device to help visualize cancerous tissue in the boundaries of a surgical site or to visualize markers and/or exosomes bearing markers in a fluid of the body. As such, in various embodiments, a method of evaluating an oncological surgical site herein can include applying a visualization device to the oncological surgical site, wherein the visualization device can include a fibrous substrate 202, and a synthetic polymer, wherein the synthetic polymer exhibits specific binding to an oncological marker. The synthetic polymer can be integrated on or into the fibrous substrate 202. The method can also include applying a visualization element 302, wherein the visualization element 302 can be bound to at least one of the synthetic polymer and the fibrous substrate 202, and then imaging the visualization device.

As previously referenced, monitoring devices herein can take on many different forms. In some embodiments, the monitoring device 500 can be substantially deformable or a deformable amorphous mass. This can allow the monitoring device to fit in various places and/or to conform to portions of the human anatomy within the body or on the surface thereof.

Referring now to FIG. 6, a schematic view of a monitoring device is shown in accordance with various embodiments herein. FIG. 6 shows an implantable monitoring device 500 and an exosome 100 bonded thereto. The implantable monitoring device 500 is in the form of a deformable amorphous mass 602. In some embodiments, the implantable monitoring device 500 can include a porous boundary layer or porous housing defining an outer surface of the device. The porous boundary layer or housing can serve to hold a sensing substrate formed of a synthetic polymer with specific binding characteristics therein (whether in a fibrous form, as a porous monolith, as discrete particles, a collection of fused or sintered particles, etc.). In various embodiments, the porous boundary layer or housing can include a porous membrane. In some embodiments, the porous boundary layer or housing can include a flexible polymeric material. In various embodiments, the porous boundary layer or housing can include a flexible pouch.

In various scenarios, it can be useful to be able to evaluate a tissue or fluid in situ within the body for the presence of a disease state marker and/or toxic substances. Sometimes, a biopsy may be taken of a tissue for further analysis in vitro. However, biopsies pose various risks. As such, in various embodiments herein, a biopsy device is included that can be used to evaluate a tissue or fluid without the need to remove a portion of tissue.

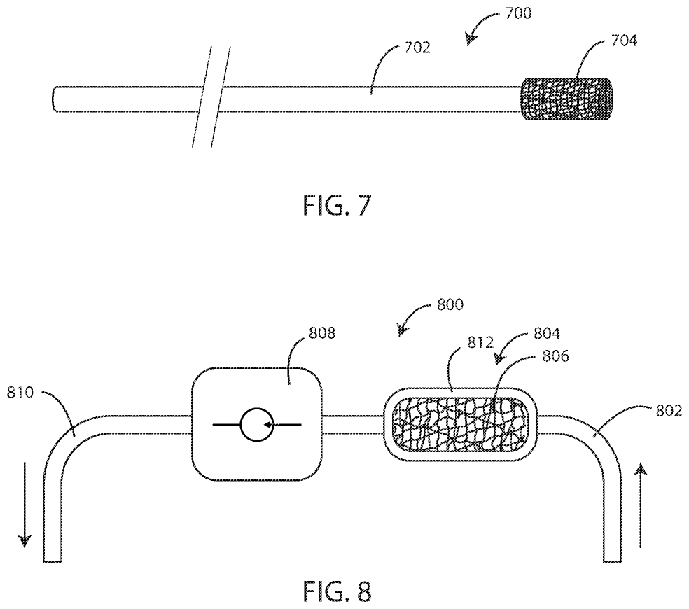

Referring now to FIG. 7, a schematic view of a biopsy device 700 is shown in accordance with various embodiments herein. The biopsy device 700 includes a shaft 702 and a tissue sampling element 704 disposed on the shaft 702. The biopsy device 700 can be inserted into a patient in order to facilitate evaluating a tissue and/or fluid thereof. The biopsy device 700 can be inserted in various ways. In some embodiments, the biopsy device 700 can be insert into an orifice of a patient. In some embodiments, the biopsy device can be inserted into an incision or a surgical site. In various embodiments, the shaft 702 can be configured to fit within an introducer sheath for intravenous insertion.

The tissue sampling element 704 can include a synthetic polymer. The synthetic polymer exhibits specific binding with at least one of a disease state marker, an exosome 100 bearing the disease state marker, and a toxic substance. In various embodiments, the tissue sampling element 704 can include a fibrous substrate 202. In various embodiments, the tissue sampling element 704 can include a microporous membrane. In various embodiments, the tissue sampling element 704 can include a microporous monolithic structure.

In various embodiments, the shaft 702 can be a flexible shaft, such as a flexible polymeric shaft. However, in some embodiments, the shaft 702 can be substantially rigid. The shaft can be formed of a polymer, a metal, a glass or ceramic, a composite, or the like.

In various embodiments, extracorporeal medical devices are included herein that can be used for evaluating tissues and/or fluids of a patient. In various embodiments, extracorporeal medical devices are included herein that can be used to remove substances, such as diseased materials or fluids, cancerous materials or fluids, toxic substances, and/or chemotherapeutic agents from a patient. For example, the device can receive fluids from the patient, remove the components targeted for removal, then return the cleaned fluids back to the patient.

Referring now to FIG. 8, a schematic view of an extracorporeal medical device 800 is shown in accordance with various embodiments herein. The extracorporeal medical device 800 includes a sampling tube 802. A fluid or other material from a patient can be drawn into the sampling tube 802. The extracorporeal medical device 800 also includes a sensing and/or removal device 804. The sensing device and/or removal device 804 can include a sensing and/or removal substrate 806 which, in some embodiments, can be disposed within a housing 812. In various embodiments, the sampling tube 802 can be in fluid communication with the housing 812.

In various embodiments, the sensing and/or removal substrate 806 can be disposed within the housing 812. In various embodiments, the sensing and/or removal substrate 806 can be configured to contact a fluid sample of a patient. The sensing and/or removal substrate 806 can include a synthetic polymer, wherein the synthetic polymer exhibits binding specificity with the substance to be removed. In some embodiments, the sensing and/or removal substrate 806 can specifically be in the form of a fibrous substrate. However, in other embodiments, the sensing and/or removal substrate 806 can take other forms including a porous monolith, a porous membrane, discrete particles, etc.

In some embodiments, the extracorporeal medical device 800 can also include a fluid pump 808. The fluid pump 808 can cause the fluid or other material to move through the extracorporeal medical device 800. Specifically, in various embodiments, the fluid pump 808 can be configured to cause a fluid sample to flow through a housing 812. The fluid pump 808 can be of various types including a piston pump, a peristaltic pump, an impeller pump, a pneumatic pump, a hydraulic pump, and the like. In various embodiments, the extracorporeal medical device 800 can also include a fluid return tube 810 in fluid communication with the housing 812. Fluids or other materials that have been cleansed of the substance(s) to be removed can be returned to the patient fluid return tube 810.

Thus, in various embodiments, a method of removing a substance from fluids of a patient can include withdrawing a fluid sample from the patient and contacting the fluid sample with an extracorporeal filtration device. The extracorporeal filtration device can include a fibrous substrate. The fibrous substrate can include a synthetic polymer, wherein the synthetic polymer exhibits binding specificity with the substance to be removed. The method can further include returning filtered fluids to the patient.

In various embodiments, a substance to be removed can include at least one of a toxic substance or a chemotherapy drug. However, in some embodiments, the substance to be removed can include a disease state marker or a material including a disease state marker. In some embodiments, the substance to be removed can include an oncological marker or an oncological material such as cancerous cells or cells bearing oncological markers.

It will be appreciated, however, that in various embodiments fluids and/or materials from a patient are not returned to the patient. For example, the fluids and/or materials can be removed from the patient and then analyzed or otherwise visualized and then simply discarded instead of being returned to the patient.

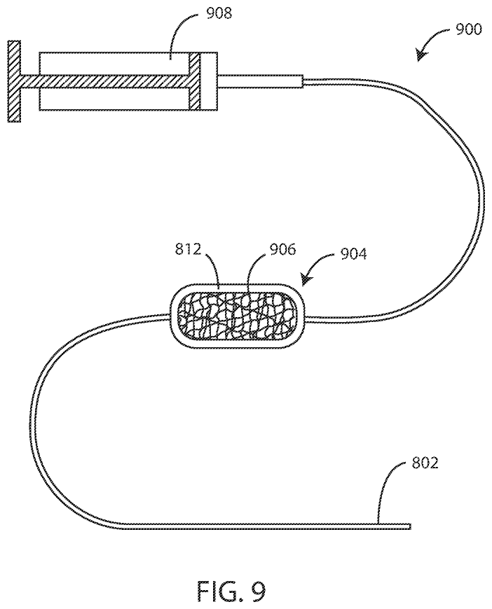

Referring now to FIG. 9, a schematic view of another embodiment of an extracorporeal medical device 900 is shown in accordance with various embodiments herein. In this embodiment, the extracorporeal medical device 900 includes a sampling tube 802 as before. The extracorporeal medical device 900 also includes a sensing device 904. The sensing device 904 includes a housing 812 and a sensing substrate 906 disposed therein. The extracorporeal medical device 900 also includes a fluid pump or suction device 908 in order to pull fluids through the sampling tube 802 and the sensing device 904. In use, fluids can be pulled through the sensing device 904 and then the sensing device 904 can be imaged and/or otherwise evaluated to detect the presence of components specifically bound to the sensing substrate 906 disposed therein.

In some embodiments, the device herein can be configured in order to be implanted within a vein, an artery, a lymph duct, a portion of a biliary duct, or another substantially tubular structure inside the body. In some cases, the device can be implanted and, while implanted, can specifically bind with components of interest (such as disease markers, toxic substances or the like). Then the device can be evaluated and/or imaged for the presence of component that are specifically bound. For example, the device can be removed from the patient and then subjected to various imaging techniques to identify the presence of a component that is specifically bound.

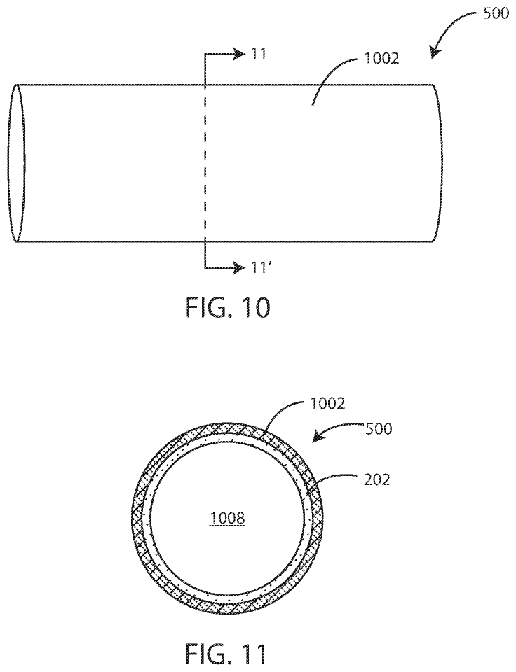

Referring now to FIG. 10, a schematic view of an implantable monitoring device 500 is shown in accordance with various embodiments herein. The implantable monitoring device 500 includes an implantable housing 1002. In some embodiments, the implantable housing 1002 can be expandable (such as balloon-expandable) so that it can expand to fit against the walls of an artery, vein, duct, tract, or the like. In some embodiments, the implantable housing 1002 can include an expandable frame structure including one or more struts such as a stent frame that can be expanded. In some embodiments, the implantable housing 1002 can define a number of apertures (not shown in this view). In some embodiments, the implantable housing 1002 can be substantially porous. In some embodiments, the implantable housing 1002 can be rigid. In some embodiments, the implantable housing 1002 can be flexible. The implantable housing 1002 can be formed of various materials including, but not limited to, a polymer, a metal, a ceramic, a composite or the like.

Referring now to FIG. 11, a cross-sectional view of an implantable monitoring device 500 as taken along line 11-11′ of FIG. 10 is shown in accordance with various embodiments herein. As before, the implantable monitoring device 500 includes an implantable housing 1002. In various embodiments, the implantable housing can include an inner surface. Within the implantable housing 1002 is a sensing/binding substrate such as a fibrous substrate 202. In various embodiments, the fibrous substrate 202 can line the inner surface of the implantable housing 1002.

In some embodiments, the implantable housing 1002, and the fibrous substrate 202 therein, can define a flow channel 1008. The flow channel 1008 can be of various sizes. In some embodiments, the diameter of the flow channel 1008 can be greater than or equal to 0.5 mm, 1.4 mm, 2.3 mm, 3.2 mm, 4.1 mm, or 5.0 mm. In some embodiments, the diameter can be less than or equal to 30.0 mm, 25.0 mm, 20.0 mm, 15.0 mm, 10.0 mm, or 5.0 mm. In some embodiments, the diameter can fall within a range of 0.5 mm to 30.0 mm, or 1.4 mm to 25.0 mm, or 2.3 mm to 20.0 mm, or 3.2 mm to 15.0 mm, or 4.1 mm to 10.0 mm, or can be about 5.0 mm.

However, in other embodiments, the fibrous substrate 202 (or another type of substrate described herein such as a porous monolith) can substantially fill the inside of the implantable housing 1002. For examples, the fibrous substrate 202 can be packed within the implantable housing 1002 like a chromatography column can be packed with media. In such an embodiment, because of the porous nature of the fibrous substrate 202, fluids and/or materials can still pass through the implantable housing 1002.

In various embodiments, the implantable monitoring device 500 can be evaluated and/or imaged for the presence of component that are specifically bound. For example, the implantable monitoring device 500 can be removed from the patient and then subjected to various imaging techniques to identify the presence of a component that is specifically bound.

As described at various points herein, substrates for purposes of sensing and/or removal of components are not just limited to fibrous substrates. For example, sensing and/or removal substrates can also include porous membranes, porous monoliths (which can include particulates bonded together to form a monolithic mass), and the like. Such structures can be formed in various ways. In some embodiments, particulates can be deposited and then bonded together. Bonding can occur in various ways. In some embodiments, a bonding agent or a cross-linking agent can be used to bond particulates together. In some embodiments, thermal treatment(s) can be used to bond particulates together. In some embodiments, ultrasonic treatment(s) can be used to bond particulates together. Many different techniques are contemplated herein.

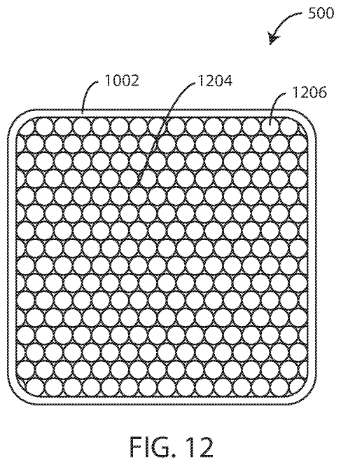

Referring now to FIG. 12, a schematic view of a monitoring device 500 (implantable or otherwise) is shown in accordance with various embodiments herein. The monitoring device 500 includes a housing 1002. A sensing substrate can be present in the form of a microporous monolith 1204. The microporous monolith 1204 can be formed of a plurality of particles 1206 that are bonded together.

### Methods

Many different methods are contemplated herein, including, but not limited to, methods of making, methods of using, methods of sensing and/or monitoring, methods of testing, methods of removing substances, methods of treating, and the like. Aspects of system/device operation described elsewhere herein can be performed as operations of one or more methods in accordance with various embodiments herein.

In an embodiment, a method of testing a patient for a disease state is included, the method can include withdrawing a fluid sample from the patient, and contacting the fluid sample with an extracorporeal monitoring device. The extracorporeal monitoring device can include a fibrous substrate. The fibrous substrate can include a synthetic polymer, wherein the synthetic polymer exhibits binding specificity with a disease state marker. The method can further include evaluating the extracorporeal monitoring device for the presence of the disease state marker.

In an embodiment of the method, the disease state marker comprises at least one of an oncological marker, a cardiovascular disease marker, and a neurological disease marker.

In an embodiment, the fibrous substrate can include electrospun fibers. In an embodiment, the fibrous substrate can include fibers of electrospun hydrogel. In an embodiment, the fibrous substrate can include fibers of electrospun crosslinked hydrogel. In an embodiment, the synthetic polymer can include a hydrogel. In an embodiment, the synthetic polymer can include a copolymer including n-isopropyl acrylamide and acrylic acid as well as other monomers.

In an embodiment, the extracorporeal monitoring device can include a flow channel. In an embodiment of the method, the fibrous substrate is disposed within or surrounds the flow channel.

In an embodiment, a method of testing a patient for a disease state is included. The method can include withdrawing a fluid sample from the patient and contacting the fluid sample with an extracorporeal monitoring device. The extracorporeal monitoring device can include a microporous membrane. The microporous membrane can include a synthetic polymer, wherein the synthetic polymer exhibits binding specificity with a disease state marker. The method can also include evaluating the extracorporeal monitoring device for the presence of the disease state marker.

In an embodiment of the method, the disease state marker comprises at least one of an oncological marker, a cardiovascular disease marker, and a neurological disease marker.

In an embodiment, the extracorporeal monitoring device can include a flow channel. In an embodiment of the method, the microporous membrane is disposed within or surrounds the flow channel.

In an embodiment, a method of testing a patient for cancer is included, the method can include withdrawing a fluid sample from the patient and contacting the fluid sample with an extracorporeal monitoring device. The extracorporeal monitoring device can include a fibrous substrate. The fibrous substrate can include a synthetic polymer. The synthetic polymer exhibits binding specificity with an oncological marker. The method can further include evaluating the extracorporeal monitoring device for the presence of at least one of the oncological marker and an exosome bearing the oncological marker. In an embodiment of the method, the oncological marker comprises at least one of VEGF, CD63, annexin A6, and mesothelin.

In an embodiment, the extracorporeal monitoring device can include a flow channel. In an embodiment of the method, the fibrous substrate is disposed within or surrounds the flow channel.

In an embodiment, a method of removing a substance from fluids of a patient is included. The method can include withdrawing a fluid sample from the patient and contacting the fluid sample with an extracorporeal filtration device. The extracorporeal filtration device can include a fibrous substrate. The fibrous substrate can include a synthetic polymer, wherein the synthetic polymer exhibits binding specificity with the substance to be removed. The method can further include returning filtered fluids to the patient. In an embodiment, the substance to be removed can include at least one of a toxic substance or a chemotherapy drug.

In an embodiment, a method of evaluating an oncological surgical site is included, the method applying a visualization device to the oncological surgical site, the visualization device can include a fibrous substrate, and a synthetic polymer, wherein the synthetic polymer exhibits specific binding to an oncological marker. The synthetic polymer can be integrated on or into the fibrous substrate. A visualization element can also be included, wherein the visualization element can be bonded to at least one of the synthetic polymer and the fibrous substrate. The method can further include imaging the visualization device.

In an embodiment of the method, the oncological marker comprises at least one of VEGF, CD63, annexin A6, and mesothelin, or exosomes bearing the same. In an embodiment, the visualization element can include a fluorescent dye. In an embodiment of the method, imaging the visualization device further comprises applying ultraviolet light. In an embodiment of the method, imaging the visualization device can include identifying exosomes bound to the visualization device.

In an embodiment of the method, the visualization device is a flexible, planar article. In an embodiment of the method, the visualization device is a flexible pouch. In an embodiment of the method, the visualization device is a deformable amorphous mass.

In an embodiment, a method of monitoring cancerous tumor progression is included, the method can include placing an implantable monitoring device within a patient. The implantable monitoring device can include a fibrous substrate, and a synthetic polymer, wherein the synthetic polymer exhibits specific binding to an oncological marker. The synthetic polymer can be integrated on or into the fibrous substrate. The implantable monitoring device can define a fluid flow channel and the synthetic polymer can be disposed within the flow channel. The method can further include removing the implantable monitoring device from the patient and analyzing the implantable monitoring device for the presence of the oncological marker.

In an embodiment, the method can further include analyzing the implantable monitoring device for the presence of exosomes bearing the oncological marker. In an embodiment of the method, the implantable monitoring device defines a lumen.

In an embodiment, a method of evaluating a tissue of a patient is included, the method can include placing a biopsy device within a patient. The biopsy device can include a shaft and a tissue sampling element. The tissue sampling element can include a fibrous substrate. The fibrous substrate can include a synthetic polymer, wherein the synthetic polymer exhibits specific binding to a disease state marker. The method can further include removing the biopsy device from the patient and analyzing the tissue sampling element for the presence of at least one of the disease state marker and an exosome bearing the disease state marker.

### Disease State Markers

Various embodiments herein include a component of a device specifically binding to a disease state marker or an exosome bearing the same. For example, methods of detecting, methods of monitoring, methods of removing can all including a component of a device specifically binding to a disease state marker or an exosome bearing the same. Similarly, various embodiments of devices herein can include a component (such as that formed at least partially from a synthetic polymer as described herein) that specifically binds to a disease state marker or an exosome bearing the same. Further details about exemplary disease state markers are provided as follows. However, it will be appreciated that this is merely provided by way of example and that further variations are contemplated herein.

In various embodiments, the disease state marker can include at least one of an oncological marker, a cardiovascular disease marker, and a neurological disease marker

In various embodiments, the disease state marker can specifically include an oncological marker. Oncological markers herein can include both circulating oncological markers as well as tumor tissue markers. Examples of oncological markers can include calcitonin, CA-125, beta-2-microglobulin, prostate specific antigen (PSA), prostate specific membrane antigen (PSMA), estrogen receptor, progesterone receptor, EGFR, PD-L1, and the like. In various embodiments, the oncological marker can further specifically include at least one of VEGF, CD63, annexin A6, and mesothelin.

Cardiovascular disease markers can specifically include one or more of CRP, ST2, TNF-alpha, GDF-15, FAS (APO-1), LP-A2, YKL-40, IL-1, osteoprotegerin, pentraxin, procalcitonin, various cytokines, serin protease PR3, soluble endoglin, adiponectin, norepinephrine, renin, angiotensin II, aldosterone, vasopressin, endothelin-1, urocortin, chromogranin A and B, MR-proADM, MMP-2, MMP-3, MMP-8, TIMP 1, IL-6, N-terminal collagen type III peptide, myostatin, syndecan-4, galectin-3, troponin T, myosin light-chain kinase 1, heart-type FA binding protein, CKMB, sFAS, HSP 60, sTRAIL, BNP, NT-proBNP, MR-proANP, sST2, GDF-15, oxidized LDL, MPO, urinary biopyrrins, urinary and plasma isoprostanes, urinary 8-hydroxyl-2′-deoxyguanosine, plasma malondialdehyde, and the like.

Neurological disease markers can specifically include one or more of IL-6, neuron-specific enolase, S100b, myelin basic protein, glial fibrillary acidic protein, B-type NGF, NMDA receptor autoantibodies, Park 7, nucleotide diphosphate kinase A, von Willebrand factor, cellular fibronectin, soluble VCAM-1, fibrinogen, soluble glycoprotein V, CRP, TNF-alpha, MMP-9, monocyte chemotactic protein-1, VCAM, and the like.

### Toxic Substances

Various embodiments herein include detecting and/or removing one or more toxic substances and/or devices for accomplishing the same. Further details about the toxic substance are provided as follows. However, it will be appreciated that this is merely provided by way of example and that further variations are contemplated herein.

In various embodiments, the toxic substance can include a chemotherapeutic agent. Chemotherapeutic agents can include, but are not limited to, alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids, vinca alkaloids, and the like.

Chemotherapeutic agents can specifically include, but are not limited to, pyrimidine antimetabolites such as 5-fluorouracil (5FU), tegafur, carmofur, doxifluridine, broxuridine, cytarabine, enocitabine, hydroxypyridine, hydroxycarbamide, methotrexate, fludarabine phosphate and the like; purine antimetabolites such as 6-mercaptopurine, 6-thioguanine, thioinosine, gemcitabine hydrochloride etc., and the like; cisplatin, carboplatin, nedaplatin, oxaliplatin and the like; nitrogen mustard alkylating agents such as nitrogen mustard, nitrogen mustard N-oxide, chlorambucil and the like; ethylenimine derivatives such as carboquone, thiotepa and the like; sulfonates such as busulfan, improsulfan tosylate and the like; nitrosourea derivatives such as nimustine hydrochloride etc., and the like; mitomycin C, bleomycin, peplomycin, daunorubicin, aclarubicin, doxorubicin, pirarubicin, THP-adriamycin, 4′-epidoxorubicin, epirubicin and the like; chromomycin A3, actinomycin D and the like; vinca alkaloids such as vinblastine, vincristine, vindesine and the like; epipodophyllotoxins such as etoposide, teniposide and the like; taxane alkaloids such as paclitaxel, docetaxel etc., and the like; molecule target therapeutic agents including imatinib, gefitinib, erlotinib, vandetanib, sunitinib, sorafenib, rituximab, cetuximab, infliximab, trastuzumab, bevacizumab, and the like

### Synthetic Polymers

Various embodiments herein include a synthetic polymer. Further details about the synthetic polymer are provided as follows. However, it will be appreciated that this is merely provided by way of example and that further variations are contemplated herein.

In various embodiments, the synthetic polymer can include a hydrogel. In various embodiments, the synthetic polymer can include a copolymer including n-isopropyl acrylamide, acrylic acid, methacrylic acid, and/or derivatives thereof.

In various embodiments, the synthetic polymer can include a copolymer including N-isopropylacrylamide (NIPAm), N-tert-butylacrylamide (TBAm); N,N′-methylenebisacrylamide (Bis), and a sulfated monomer. In some embodiments, the sulfonated monomer is 2-acrylamido-2-methylpropane sulfonic acid (AS). In some embodiments, the sulfated monomer is an N-acetylglucosamine (GlcNAc). In various embodiments, the sulfated monomer is an isomer of GlcNAc, including but not limited to 3 S-GlcNAc, 45-GlcNAc, or 65-GlcNAc. In one embodiment, the sulfated monomer is 3,4,65-GlcNAc.

In some embodiments, the polymer can include a copolymer or terpolymer including at least two monomers selected from: N-t-butylacrylamide (TBAm), acrylic acid (AAc), N-isopropylacrylamide (NIPAm), N,N′-methylenebis(acrylamide) (MBAm), N,N′-ethylenebis(acrylamide) (EBAm), acrylamide (AAm), 1-vinyl imidazole (VI), N-(3-aminopropyl)acrylamide (APAm), N-phenyl acrylamide (PAm), N-[2-[[[5-(Dimethylamino)-1-naphthalenyl]sulfonyl]-aminolethyl1-2-propenamide (DANSAm), fluorescein o-acrylate (FAc), polyethylene glycol diacrylate (PEGDAc), N-t-butylmethacrylamide (TBMAm), methacrylic acid (MAAc), N-isopropylmethacrylamide (NIPMAm), N,N′-methylenebis(methacrylamide) (MBMAm), N,N′-ethylenebis(methacrylamide) (EBMAm), methacrylamide (MAAm), N-(3-aminopropyl)methacrylamide (APMAm), N-phenyl methacrylamide (PMAmN-[2-[[[5-(Dimethylamino)-1-naphthalenyl]sulfonyl]-amino]ethyl]-2-methyl-Z-propenamide (DANSMAm), fluorescein o-methacrylate (FMAc) and polyethylene glycol dimethacrylate (PEGDMAc).

In some embodiments, the synthetic polymer can be “imprinted” in order to provide specific binding characteristics. An imprinted polymer can be formed using a physical template corresponding to the desired specific binding target. But, in other embodiments, the synthetic polymer can be provided with specific binding characteristics without imprinting. For example, specificity can be provided based on conformational promiscuity that allows for optimization of complementary interactions with target molecule surfaces by an induced fit. Further, specificity for particular targets can be achieved by adjusting the identity and amounts of monomers used to form the copolymer resulting in a lightly cross-linked network polymer presenting 3-dimensional arrays of linear polymer segments that can serve as both continuous and discontinuous recognition elements for binding with target surfaces.

However, in some embodiments, the synthetic polymer can include imprinted polymers that are polymerized in the presence of a target molecule for specific binding (e.g., specific binding to a disease state marker, a toxic substance, and the like), wherein the imprinted polymer comprises any of the previously described monomers, wherein the resulting copolymer exhibits specific binding for the target molecule. Such synthetic polymers can be formed in various way. In some embodiments, a method of making an imprinted synthetic polymer can include forming a mixture of monomers along with target molecules in an aqueous medium and incubating the reaction mixture under polymerization conditions such that imprinted synthetic polymers are generated that are specific for the target molecules.

In further embodiments, the imprinted polymer can further include a crosslinking agent. In some embodiments, the crosslinking agent comprises N,N′-methylenebis(acrylamide) or similar reagent. In some embodiments, the crosslinking agent can be activated only after an electrospinning process has taken place.

Further details of exemplary synthetic polymers with specific binding characteristics are described in U.S. Publ. Pat. Appl. No. 20190216744; U.S. Pat. No. 9,173,943; and U.S. Publ. Appl. No. 2012/0097613, the contents of all of which are herein incorporated by reference.

Aspects may be better understood with reference to the following examples. These examples are intended to be representative of specific embodiments, but are not intended as limiting the overall scope of embodiments herein.

### EXAMPLES

### Example 1: Nanoparticle (NP) Synthesis

Nanoparticles optimized to adsorb melittin (a peptide consisting of 26 amino acids) were synthesized according to the following procedure. N-Isopropylacrylamide (195 mg, 1.72 mmol) was weighed into a 50-mL centrifuge tube. t-Butylacrylamide (165 mg, 1.3 mmol) was added to the tube followed by 1 mL absolute ethanol. All solids were dissolved with gentle swirling. After dissolution, 40 mL of water was added to the tube. N, N′-Methylenebisacrylamide (10 mg, 0.065 mmol) was dissolved in 2 mL of water in a separate vial and added to the previously prepared solution. Acrylic acid (11 μL, 0.16 mmol) and 1 mL of a 1% wt/vol aqueous solution of sodium dodecyl sulfate were added next. Water was added to the tube to bring the total solution volume up to 50 mL. The tube was capped and inverted gently several times to mix the contents. The contents of the tube were transferred to a 100-mL, two-neck, round-bottom flask. Both necks were sealed with septa and, using nitrogen introduced via a syringe needle, the solution was deoxygenated over the course of 30 minutes. Separately, a solution of ammonium persulfate (150 mg, 0.65 mmol) in 3 mL water was prepared. This solution was also purged with nitrogen for 30 minutes, then 0.6 mL of the solution was transferred to the round-bottom flask via disposable syringe. One of the septa on the flask was replaced with a thermocouple adapter and thermocouple. A magnetic stirring pea was placed in the flask. Using a temperature controller, the flask was heated to 60° C. for three hours. The initially clear solution became cloudy as the nanoparticles formed during polymerization.

### Example 2: Nanoparticle Purification

Nanoparticle purification was based on the procedure described in Shea (Nature Protocols, 10 (4) 2015, pp. 595-604). The aqueous nanoparticle suspension was transferred to a dialysis bag with a molecular weight cutoff of 12-14 kDa. The suspension was dialyzed for 4 days against 4 L of water at room temperature with stirring. The water was changed twice per day.

### Example 3: Evaluation of Melittin Affinity of the Nanoparticles by Bovine Red Blood Cell (RBC) Assay

The RBC assay was also based on the literature procedure found in Nature Protocols, 10 (4) 2015, pp. 595-604. A 6% suspension of washed bovine RBCs was prepared in PBS buffer. A test suspension of the dialyzed nanoparticles and melittin solution (at a concentration of 18 μM) in 10×PBS was incubated at 37° C. in a block heater for 30 minutes. Control suspensions were also prepared and incubated. The compositions of the various test solutions and suspensions are listed in the table. After incubation, all of the suspensions were centrifuged at 3,200 g for five minutes. The hemolytic effect of melittin results in a deep red supernatant after the suspension is spun down. A colorless supernatant demonstrates the absence of melittin activity. The results from testing of the various RBC suspensions are collected in the table below.

| 10x PBS | | | | | |
|---|---|---|---|---|---|
| buffer | Melittin | Nanoparticles | DI water | Supernatant | Comments |
| 100 μL | None | None | 400 μL | Colorless | Negative control |
| 100 μL | 200 μL | None | 700 μL | Red | Positive control |
| 100 μL | 200 μL | 700 μL | None | Colorless | NP test |
| | | | | | suspension |

### Example 4: Synthesis of Linear Polymer with Melittin Affinity

A soluble, linear polymer with the same comonomer composition as the nanoparticles optimized to adsorb melittin, minus the diacrylate crosslinker, was synthesized by the following procedure. N-Isopropylacrylamide (15.2 g, 134 mmol) was weighed into a beaker, followed by 40 mL of N,N-dimethylformamide (DMF), t-butylacrylamide (13.2 g, 104 mmol) and acrylic acid (880 μL, 0.925 g, 12.8 mmol). All monomers were dissolved with gentle swirling. DMF was added to the beaker to bring the total solution volume up to 50 mL. The contents of the beaker were swirled gently to mix the contents. The resulting solution transferred to a 100-mL, two-neck, round-bottom flask. Both necks were sealed with septa and, using nitrogen introduced via a syringe needle, the solution was deoxygenated over the course of 30 minutes. Separately, a solution of ammonium persulfate (60 mg, 0.26 mmol) in 4 mL DMF was prepared. This solution was also purged with nitrogen for 30 minutes, then the solution was transferred to the round-bottom flask via disposable syringe. One of the septa on the flask was replaced with a thermocouple adapter and thermocouple. A magnetic stirring pea was placed in the flask. With the aid of a temperature controller, the flask was heated to 60° C. with stirring for three hours. A polymer solution with visibly high viscosity was produced.

### Example 5: Electrospinning of Linear Polymer with Melittin Affinity

A polymer with melittin affinity was electrospun to verify that the polymer was capable of electrospinning. In specific, a 35% solids solution suitable for electrospinning was prepared by diluting 10 mL of a 41% solids DMF solution of polymer with 2.5 mL of acetone. Similarly, a 25% solids solution suitable for electrospinning was prepared by diluting 10 mL of a 41% solids DMF solution of polymer 3.1 mL of DMF and 3.3 mL of acetone. Both solutions were mixed until homogeneous using a Flacktek SpeedMixer. After mixing, a portion of the diluted solution was loaded into a 5 mL disposable syringe with a 16-ga needle. The syringe was secured in an electrospinning apparatus. Electrospinning was carried out at a flow rate of 0.5 mL/h and a potential of 4,000 volts. Voltage was adjusted during the electrospinning process in order to maintain a stable Taylor cone. Fibers were collected on an aluminum foil-wrapped steel mandrel. The diameter of the fibers obtained from the 35% solids solution was approximately 3 μm, as determined by electron microscopy. Fibers obtained from the 25% solids solution were approximately 1 μm in diameter.

### Example 6: Evaluation of the Melittin Affinity of Electrospun Fibers by Bovine Red Blood Cell (RBC) Assay

The electrospun polymer with melittin affinity was then tested to verify that it maintained desirable binding properties. In specific, the RBC assay used in Example 3 was performed using electrospun fibers as the test adsorbent for melittin. A control suspension was also prepared and incubated.

Electrospun fibers (about 25 mg) from Example 5 were soaked in deionized water for an hour prior to the assay in order to any residual DMF, acetone or unreacted monomers. After rinsing, each fiber sample was placed in a small vial with 1 mL of 18 μM melittin. The vials containing fibers and melittin solution were attached to the shaft of an overhead stirrer mounted horizontally and spun at 60 rpm for one hour. The RBC assay was then performed on these solutions. The results from testing of the various RBC suspensions are collected in the table below.

| PBS | | | | |
|---|---|---|---|---|
| buffer | Melittin | E-spun fibers | Supernatant | Comments |
| Y | N | N | Colorless | Negative control |
| Y | Y | N | Red | Positive control |
| Y | Y | Y-Spun from 35% | Colorless | Test of fiber affinity for |
| | | solution | | melittin |
| Y | Y | Y-Spun from 25% | Colorless | Test of fiber affinity for |
| | | solution | | melittin |

This example shows that the polymer maintained desirable binding properties even after being electrospun.

### Example 7: Crosslinking of Electrospun Fibers with Melittin Affinity

Integration into a device frequently requires sufficient material durability. As such, the electrospun polymer with melittin affinity was cross-linked to improve its durability and then compared with un-cross-linked material. In specific, a portion of an electrospun fiber mat (from a 25% polymer solution) prepared in Example 5 was soaked for an hour in a saturated aqueous solution of calcium carbonate. Successful crosslinking was demonstrated by exposing the as-spun fibers and the crosslinked fibers to acetone, a good solvent for the polymer. The fiber mat that was uncrosslinked dissolved immediately; the crosslinked fiber mat partially dissolved.

Similarly, a portion of an electrospun fiber mat prepared in Example 5 (also from a 25% polymer solution) was soaked for 20 minutes in a 1% hexane solution of benzophenone. The benzophenone-impregnated mat was exposed to UV light from a Dymax LED lamp used for the curing of adhesives. Successful crosslinking was demonstrated by exposing the as-spun fibers and the crosslinked fibers to acetone, a good solvent for the polymer. The fiber mat that was uncrosslinked dissolved immediately; the UV-crosslinked fiber mat did not dissolve at all. This example shows that the electrospun polymer material herein can be cross-linked to improve its durability.

### Example 8: Evaluation of the Melittin Affinity of Crosslinked Electrospun Fibers by Bovine Red Blood Cell (RBC) Assay

The electrospun polymer with melittin affinity was then tested to verify that it maintained desirable binding properties. The RBC assay used in Example 3 and 6 was performed using ionically crosslinked electrospun fibers as the test adsorbent for melittin. The results are collected in the table below.

| PBS | | Agent used to adsorb | | |
|---|---|---|---|---|
| buffer | Melittin | melittin | Supernatant | Comments |
| Y | N | None | Colorless | Negative control |
| Y | Y | None | Red | Positive control |
| Y | Y | Nanoparticles from | Colorless | Nanoparticle standard |
| | | Example 2 | | |
| Y | Y | Uncrosslinked electrospun | Colorless | Test of uncrosslinked |
| | | fibers | | fiber's affinity for |
| | | | | melittin |
| Y | Y | Crosslinked electrospun | Colorless | Test of ionically |
| | | fibers (crosslinked via | | crosslinked fiber's |
| | | CaCO₃) | | affinity for melittin |

This example shows that the polymer maintained desirable binding properties even after being electrospun and cross-linked.

### Example 9: Preparation and Evaluation of the Melittin Affinity of PVDF-HFP Electrospun Fibers with Grafted Nanoparticles in the Bovine Red Blood Cell (RBC) Assay

In accordance with some embodiments herein, a fibrous mat can be formed with electrospun polymeric fibers (exhibiting specific binding properties or not) and then polymeric nanoparticles exhibiting desirable binding properties can be attached thereto such as through covalent or non-covalent binding techniques. In this example, a small quantity of a previously electrospun fiber mat (34.0 mg) made of poly(vinylidene fluoride-co-hexafluoropropylene), or PVDF-HFP, was soaked for 10 minutes in the nanoparticle dispersion prepared in Example 1. It was then air-dried for 24 hours. The weight of the treated and dried fiber mat was 34.5 mg. The fiber mat was next exposed to plasma under vacuum to generate charged groups on the polymeric materials to non-covalently bind the nanoparticles to the fiber mat. The plasma-treated PVDF-HFP fiber mat, in contrast to the untreated mat, demonstrated an ability to adsorb melittin, with results as listed in the table below.

| PBS | | Agent used to | | |
|---|---|---|---|---|
| buffer | Melittin | adsorb melittin | Supernatant | Comments |
| Y | N | None | Colorless | Negative control |
| Y | Y | None | Red | Positive control |
| Y | Y | Nanoparticles from | Colorless | Nanoparticle standard |
| | | Example 2 | | |
| Y | Y | Electrospun PVDF-HFP | Red | Test of non-grafted PVDF- |
| | | fiber mat | | HFP fiber's affinity for |
| | | | | melittin |
| Y | Y | Electrospun PVDF-HFP | Colorless | Test of ionically crosslinked |
| | | fiber mat with grafted | | fiber's affinity for melittin |
| | | nanoparticles | | |

This example shows that the polymer with desirable binding properties can be grafted into a mat of electrospun fibers which is formed of a polymer without the same binding properties.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of testing a patient for a disease state comprising:
withdrawing a fluid sample from the patient; and
contacting the fluid sample with an extracorporeal monitoring device, the extracorporeal monitoring device comprising
a microporous membrane, the microporous membrane comprising a synthetic polymer, wherein the synthetic polymer exhibits binding specificity with a disease state marker; and
evaluating the extracorporeal monitoring device for the presence of the disease state marker.

2. The method of testing a patient for a disease state of claim 1 wherein the disease state marker comprises at least one of an oncological marker, a cardiovascular disease marker, and a neurological disease marker.

3. The method of testing a patient for a disease state of claim 1, wherein the disease-state marker comprises at least one of VEGF, CD63, annexin A6, and mesothelin.

4. The method of testing a patient for a disease state of claim 1, the microporous membrane comprising electrospun fibers.

5. The method of testing a patient for a disease state of claim 1, the synthetic polymer comprising a hydrogel.

6. The method of testing a patient for a disease state of claim 1, the synthetic polymer comprising a copolymer including n-isopropyl acrylamide and acrylic acid.

7. The method of testing a patient for a disease state of claim 1, the extracorporeal monitoring device comprising a flow channel.

8. A method of evaluating a tissue of a patient comprising:
placing a biopsy device within a patient, the biopsy device comprising a shaft; and
a tissue sampling element, the tissue sampling element comprising
a fibrous substrate, the fibrous substrate comprising a synthetic polymer, wherein the synthetic polymer exhibits specific binding to a disease state marker;
removing the biopsy device from the patient; and
analyzing the tissue sampling element for the presence of at least one of the disease state marker and an exosome bearing the disease state marker.

9. The method of evaluating a tissue of a patient of claim 8, wherein the disease state marker comprises an oncological marker comprising at least one of VEGF, CD63, annexin A6, and mesothelin.

10. The method of evaluating a tissue of a patient of claim 8, the synthetic polymer comprising a hydrogel.

11. The method of evaluating a tissue of a patient of claim 8, the fibrous substrate comprising electrospun fibers.

12. The method of evaluating a tissue of a patient of claim 11, wherein the electrospun fibers are formed at least partly from the synthetic polymer.

13. A method of removing a substance from fluids of a patient comprising:
withdrawing a fluid sample from the patient; and
contacting the fluid sample with an extracorporeal filtration device, the extracorporeal filtration device comprising
a fibrous substrate, the fibrous substrate comprising a synthetic polymer, wherein the synthetic polymer exhibits binding specificity with the substance to be removed; and
returning filtered fluids to the patient.

14. The method of removing a substance from fluids of a patient of claim 13, the fibrous substrate comprising electrospun fibers.

15. The method of removing a substance from fluids of a patient of claim 13, the synthetic polymer comprising a hydrogel.

16. The method of removing a substance from fluids of a patient of claim 13, the extracorporeal filtration device comprising a flow channel.

17. The method of removing a substance from fluids of a patient of claim 16, wherein the fibrous substrate is disposed within or surrounds the flow channel.

18. The method of removing a substance from fluids of a patient of claim 13, the substance comprising at least one of a toxic substance or a chemotherapy drug.

19. The method of removing a substance from fluids of a patient of claim 13, the substance comprising an oncological marker or an exosome bearing the same.

20. The method of removing a substance from fluids of a patient of claim 13, the substance comprising an agent responsible for the growth and/or spread of a cancer.
